(19) United States
(12) Patent Application Publication
(10) Pub. No.: US 2026/0317807 A1
(43) Pub. Date: Oct. 15, 2026

(54) CHEMILUMINESCENCE-INDUCED PLASMONIC CURRENT (CIPC)

(71) Applicant: UNIVERSITY OF MARYLAND, BALTIMORE COUNTY, Baltimore, MD (US)

(72) Inventor: Chris D. Geddes, Baltimore, MD (US)

(21) Appl. No.: 19/634,602

(22) Filed: Mar. 31, 2026

Related U.S. Application Data
(60) Provisional application No. 63/786,478, filed on Apr. 10, 2025.

(51) Int. Cl. C12Q 1/66; C09K 11/07; G01N 21/76

(52) U.S. Cl. CPC C12Q 1/66; C09K 11/07; G01N 21/76; C09K 2211/1007; C09K 2211/1018

(57) ABSTRACT

A method of generating and detecting an induced chemiluminescence-induced plasmonic current (CIPC) is described. CIPC is a “plasmon to current” technique which eliminates the need for traditional photodetectors and associated optics required for traditional “plasmon to light” techniques such as metal-enhanced chemiluminescence and without the use of any photoexcitation means, e.g., as used in metal-enhanced fluorescence. Advantageously, the CIPC method can be used instead of photodetectors in any process that detects and measures chemiluminescence.

### CROSS REFERENCE TO RELATED APPLICATIONS

This application claims priority to U.S. Provisional Patent Application No. 63/786,478 filed on Apr. 10, 2025 in the name of Chris D. Geddes entitled “CHEMILUMINESCENCE-INDUCED PLASMONIC CURRENT (CIPC),” which is hereby incorporated by reference herein in its entirety.

### FIELD OF THE INVENTION

This invention relates to a system and method of detecting a Chemiluminescence Induced Plasmonic Current (CIPC) and using same in chemiluminescence assays.

### BACKGROUND OF THE INVENTION

Chemiluminescence assays are well known in the art as analytical detection methods that exploit the emission of light resulting from a chemical reaction to quantify the presence or concentration of a target analyte in a sample. In a typical chemiluminescence system, a luminescent substrate, e.g., luminol, acridinium esters, or dioxetane derivatives, undergoes an oxidation reaction, often catalyzed by an enzyme (e.g., horseradish peroxidase or alkaline phosphatase) or facilitated by a chemical oxidant (e.g., hydrogen peroxide), producing an excited-state intermediate that relaxes to its ground state with the concomitant emission of photons. The emitted light is measured by a photodetector, such as a photomultiplier tube or a charge-coupled device, and the signal intensity is correlated to the quantity of analyte present. These assays are widely employed in immunoassay platforms (including enzyme-linked immunosorbent assays adapted for chemiluminescent detection), nucleic acid hybridization assays, and point-of-care diagnostic devices, owing to their simplicity, broad dynamic range, reduced background interference, and the absence of a requirement for an external excitation light source, which distinguishes them from fluorescence-based detection methodologies.

The instant inventors previously introduced Metal-Enhanced Fluorescence (MEF), which is a phenomenon that is defined by a marked increase in the emissive properties of a fluorophore when brought into the near-field of a plasmonically-active nanoparticle. When the fluorophore and the plasmonically-active nanoparticle are in close proximity to each other, the fluorophore dipole is mirrored in the metal surface plasmons and the system is coupled together in this excited-state, allowing ultimately for a new method of excited state relaxation. This allows the system to emit photons faster, leading to a decreased lifetime of the system, and an overall increase in fluorescence intensity. This phenomenon is not just limited to fluorescence, but also to other excited states and reactions, including near-field interactions of chemiluminescence. For example, Metal-Enhanced Chemiluminescence (MEC) is described in the literature in an analogous manner as MEF. Some of the pathways for chemiluminescence involve a fluorophore that is chemically excited in solution, not using an external pumping light source like in MEF, but instead using a high energy intermediate compound that is able to donate energy to the fluorophore allowing it to reach its excited state and ultimately produce visible light. Another pathway involves a non-fluorescent chemical that is activated via a chemical reaction which produces light. It has been shown that in both of these pathways the interaction with the plasmonic nanoparticles leads to a greater intensity of visible light from the sample, i.e., MEC, as compared to the situation where no nanoparticles are present.

Disadvantageously, all of the chemiluminescent analyses and assays of the prior art require moderately expensive chemiluminescence plate readers to detect the far-field emissions. Further, the chemiluminescence emission intensity from the actual assay solutions tends to be very weak. There continues to be a need in the art for methods of maximizing a luminescence yield that would benefit overall detectability of a target analyte and increase the sensitivity of assays. Towards that end, an improved system and method of detection is disclosed herein, CIPC, which doesn't suffer from the disadvantages of the prior art because the CIPC system and method doesn't detect radiative emission, instead measuring current induced in a metal nanoparticle film. The CIPC system and method described herein is simple, cost-effective, easy to use, and has a high linear detection range for any application.

### SUMMARY OF THE INVENTION

In some aspects, a method of generating and detecting an induced chemiluminescence-induced plasmonic current (CIPC) is described, said method comprising:
positioning a substrate comprising a film of isolated metal nanoparticles between two electrodes;
introducing a solution onto the film of metal nanoparticles, wherein the solution comprises at least one chemiluminescent species; and
activating the at least one chemiluminescent species with a triggering substance, wherein energy generated by the at least one activated chemiluminescent species nonradiatively transfers energy to the film of metal nanoparticles, resulting in an induced electrical current across the film; and
applying a bias voltage across the film and detecting the induced current.

In other aspects, the method of generating and detecting a chemiluminescence-induced plasmonic current is used to detect a target molecule or biomolecule in a chemiluminescence assay.

Other aspects and advantages will be more fully apparent from the ensuing disclosure and appended claims.

### BRIEF DESCRIPTION OF THE FIGURES

FIG. 1A illustrates far-field excited fluorophore induced plasmonic current, including non-current generating quanta emitted as metal enhanced fluorescence (MEF).

FIG. 1B is a schematic of chemiluminescence induced plasmonic current, including non-current generating quanta emitted as metal enhanced chemiluminescence (MEC).

FIG. 2 is a schematic for plasmonic current detection and simultaneous detection of far-field chemiluminescent emission. Electrodes were chosen to match the metal of the nanoparticle film, for example, silver films use silver electrodes and gold films use gold electrodes.

FIG. 3 shows the orientation of chemiluminescent solution applied to a silver nanoparticle film, as well as the shape and size for “Area 1” (top right, 5 cm²), and “Area 2” (bottom right, 3 cm²). (A) in the top left, being 5 cm², illustrates the chemiluminescent solution in contact with both electrodes (labeled as “E”). (B) in middle left, being 3 cm², illustrates the chemiluminescent solution in contact with one electrode. (C) bottom left, being 3 cm², illustrates the chemiluminescent solution in contact with no electrodes.

FIG. 4 illustrates the experimental differences between (B) and (C) using diphenyl oxalate and fluorescein solutions on the metallic nanoparticle substrates. The baseline current is labeled “baseline.” The peak current after catalyst activation is labeled “peak current.” The solution excited via 473 nm laser @ 10 mW power after the chemiluminescent reaction had completed is labeled “FIPC.” The left (B) set of bars represents samples in contact with a single electrode with a 3 cm² surface area. The right (C) represents the samples not in contact with any electrode also with a 3 cm² surface area. A Bias voltage of 0.005 V was applied across the films. Resistance (B) 5.95×10⁹Ω, (C) 1.41×10¹⁰Ω, per linear cm. The distance between electrodes=1 cm. The electrodes are labeled as “E,” while the excitation spot is labeled as “S.”

FIG. 5A shows the sample orientations for (A) and (B) for FIGS. 5B and 5C.

FIG. 5B shows the peak induced current from activated Luminol solution for both 5 cm² and 3 cm² surface areas on both glass and Ag substrates. A bias voltage of 0.005 V was applied.

FIG. 5C shows the induced current from luminol per cm² for both Ag and glass substrates. A bias voltage of 0.005 V was applied.

FIG. 6A illustrates a rate of induced current decays, i.e., rate of change of current from a sulforhodamine/diphenyl oxalate solution on a gold nanoparticle film.

FIG. 6B illustrates a rate of induced current decays, i.e., rate of change of current from a sulforhodamine/diphenyl oxalate solution on a bare glass control sample.

FIG. 6C illustrates an induced current decay rates from a luminol solution on a silver nanoparticle film.

FIG. 6D illustrates an induced current decay rates from a luminol solution on a bare glass control sample.

FIG. 7 is a diagram depicting the decay of CIPC from excited fluorophores, both coupled and not coupled to the surface nanoparticles.

FIG. 8A illustrates the voltage sweeps for active luminol solution on an Ag film. Voltage sweeps were performed by initially applying 0 V, then increasing the voltage by 0.0005 V every second until 0.005 V was reached.

FIG. 8B illustrates the voltage sweeps for inactive luminol solution on a Ag film. Voltage sweeps were performed by initially applying 0 V, then increasing the voltage by 0.0005 V every second until 0.005 V was reached.

FIG. 8C is the resistance and conductivity of the luminol after activation at the 5 sec time point.

FIG. 9A upper illustrates luminescence of the luminol reaction on a 3 nm thick Cu film with a low bias voltage of 0.005 V to replicate CIPC. The lower figure illustrates peak luminescence at 493 nm plotted over time.

FIG. 9B upper illustrates luminescence of the luminol reaction on 3 nm Cu film with a high bias voltage of 5 V to replicate a voltage gated MEC experiment. The lower figure illustrates peak luminescence at 493 nm plotted over time.

FIG. 10 illustrates a luminescence spectra of 100 μL of stock concentration of luciferase enzyme with 100 μL of 100 μM ATP stock solution, wherein the solutions were mixed 5 minutes prior to testing. Testing performed on a QUANTAWELL™ silver nanoparticle lined 96-well plate to enhance the far-field emission intensity.

FIG. 11 illustrates peak CIPC values obtained from 100 μL working strength luciferase enzyme mixed with 100 μL of various concentrations of ATP working solution. Solutions were allowed to mix for 4.5 minutes, then applied to the film to observe the 5 min peak current response. The highest current value seen between 4.9 and 5.1 minutes was chosen and is reported here. Inactive solution 15 minutes after activation shown as well. Tested on a 3 nm Cu Film.

The features and advantages of the invention are more fully illustrated by the following non-limiting example, wherein all components are used in a particular form to demonstrate the usability and practice.

### DETAILED DESCRIPTION, AND PREFERRED EMBODIMENTS THEREOF

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

As defined herein, an “island-like film” is a non-continuous deposit of isolated metal nanoparticles on a clean substrate, said nanoparticles having a median diameter in a range from about 3 nm-500 nm, with median aspect ratios in a range from about 1:1 to 1:6, and the median distance of nanoparticle to nanoparticle is about 2 nm-250 nm. In some embodiments, the nanoparticles have a median diameter of about 3 nm to about 20 nm, or about 20 nm to about 40 nm, or about 40 nm to about 60 nm, or about 60 nm to about 80 nm, or about 80 nm to about 100 nm, or about 100 nm to about 120 nm, or about 120 nm to about 140 nm, or about 140 nm to about 160 nm, or about 160 nm to about 180 nm, or about 180 nm to about 200 nm, or about 200 nm to about 220 nm, or about 220 nm to about 240 nm, or about 240 nm to about 260 nm, or about 260 nm to about 280 nm, or about 280 nm to about 300 nm, or about 300 nm to about 320 nm, or about 320 nm to about 340 nm, or about 340 nm to about 360 nm, or about 360 nm to about 380 nm, or about 380 nm to about 400 nm, or about 400 nm to about 420 nm, or about 420 nm to about 440 nm, or about 440 nm to about 460 nm, or about 460 nm to about 480 nm, or about 480 nm to about 500 nm. In some embodiments, the median aspect ratio of the nanoparticles is about 1:1 to about 1:2. In some embodiments, the median aspect ratio of the nanoparticles is about 1:2 to about 1:3. In some embodiments, the median aspect ratio of the nanoparticles is about 1:3 to about 1:4. In some embodiments, the median aspect ratio of the nanoparticles is about 1:4 to about 1:5. In some embodiments, the median aspect ratio of the nanoparticles is about 1:5 to about 1:6. In some embodiments, the median distance of nanoparticle to nanoparticle is about 2 nm-10 nm apart, or about 10 nm to about 30 nm, or about 30 nm to about 50 nm, or about 50 nm to about 70 nm, or about 70 nm to about 90 nm, or about 90 nm to about 110 nm, or about 110 nm to about 130 nm, or about 130 nm to about 150 nm, or about 150 nm to about 170 nm, or about 170 nm to about 190 nm, or about 190 nm to about 210 nm, or about 210 nm to about 230 nm, or about 230 nm to about 250 nm.

In some embodiments, the island-like film is identified by the presence of a coulombic blockade in the current response or the presence of a staircase-like response to increasing voltage, indicative of a charge and discharge cycle across the film for different sized clusters of nanoparticles, electrically confirming the presence of these isolated nanoparticles and hence the island-like film. It should be appreciated that the metal nanoparticles can consist of only one metal or can comprise more than one metal, as understood by the person skilled in the art. The preferred thickness of the nanoparticle deposit will depend on the metal nanoparticles and the desired chemiluminescent species used so that there is substantial spectral overlap between the emission of the excited-state chemiluminescent species and the plasmon absorption of the metal nanoparticles, as readily understood by the person skilled in the art.

As used herein, “metal” or “metal nanoparticles” include traditionally understood “noble metals” such as silver, gold, platinum, palladium, rhodium, ruthenium, osmium, and iridium, but also less expensive transition metals including, copper, nickel, chromium, and zinc. Additionally, the metal nanoparticles may comprise aluminum. In some embodiments, the metal nanoparticles are plasmonically-active and share a strong absorption-emission spectral overlap with the desired chemiluminescent species.

As used herein, a “substrate” can include a material comprising, consisting essentially of, or consisting of glass, quartz, plastic, a semiconductor, paper, cellulose, cotton, nylon, silk, sapphire, diamond, noble metal films, wool fabrics, bank notes, indium tin oxide, metallic alloys or dielectric materials.

As used herein, electron “hopping” or “tunneling” occurs when the charging energy for a metal particle to gain an electron is less than the thermal energy of the system, according to E_{C}=e²/2C<k_{B}T, wherein E_{C} is charging energy, e is the elementary electric charge, C is capacitance of the particle and is directly proportional to the size of the metal particle, k_{B} is the Boltzmann constant, and Tis temperature. Without being bound by theory, it is believed that when a chemiluminescent solution is activated, e.g., using a triggering substance, energy radiatively transfers to the metal nanoparticles and this energy helps overcome the coulombic gap, permitting electron transport from metal nanoparticle-island to metal-nanoparticle island, i.e., island hoping, leading to increased charge transport across the metal nanoparticle film, with a concomitant increase in electrical current.

In recent years the ability of near-field fluorophores to non-radiatively couple to metal nanoparticles within plasmonic films and produce current flow has attracted notable attention. Fluorophore-induced plasmonic current (FIPC) is a detection method for excited state fluorophores, wherein nanoparticles used to elicit MEF are spaced closer together, allowing the nanoparticle to charge and then discharge its energy, the energy “hopping” or “tunneling” to an adjacent nanoparticle island and then another, and so on, generating a current flow. FIPC is in stark contrast to MEF or MEC, where the fluorophore-coupled quanta is radiated by the nanoparticle itself into the far-field as enhanced emission. The FIPC technique has been shown to be effective at detecting chemical probes and their associated analytes. Disadvantageously, any fluorescence method still requires the introduction of far-field excitation, and suffers from unwanted biological autofluorescence and generally poor fluorophore photostability. For the first time, chemically induced excited states are shown to generate notable current on films, in a new technique called Chemiluminescence Induced Plasmonic Current (CIPC). CIPC is a “plasmon to current” technique which eliminates the need for traditional photodetectors and associated optics required for traditional “plasmon to light” techniques such as MEC and without the use of any photoexcitation means, e.g., as used in MEF and FIPC.

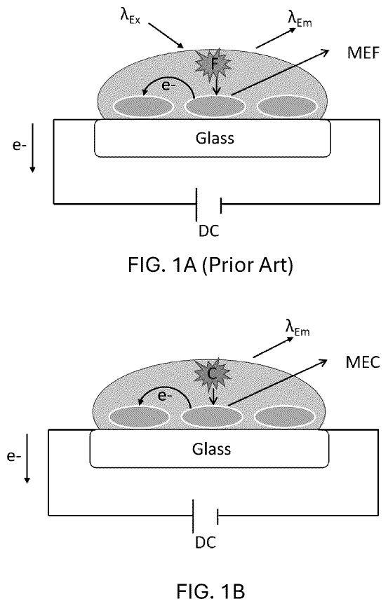

Broadly, a system and method for CIPC is disclosed herein. A diagram showing the mechanism for CIPC is detailed in FIG. 1B, including a depiction of the “tunneling” leading to induced current as well as non-current generating quanta emitted as metal enhanced chemiluminescence (MEC).

In a first aspect, a method of generating and detecting an induced chemiluminescence-induced plasmonic current (CIPC) is described, said method comprising:
positioning a substrate comprising a film of isolated metal nanoparticles between two electrodes;
introducing a solution onto the film of metal nanoparticles, wherein the solution comprises at least one chemiluminescent species; and
activating the at least one chemiluminescent species with a triggering substance, wherein energy generated by the at least one activated chemiluminescent species nonradiatively transfers energy to the film of metal nanoparticles, resulting in an induced electrical current across the film; and
applying a bias voltage across the film and detecting the induced current.

Advantageously, the induced electrical current of the at least one chemiluminescent species in the presence of the film of metal nanoparticles is enhanced relative to a control lacking the film of metal nanoparticles.

The chemiluminescent species can include, but are not limited to: luminol, which can be triggered by hydrogen peroxide in the presence of a metal catalyst or horseradish peroxidase (HRP) under alkaline conditions; lucigenin (bis-N-methylacridinium nitrate), which is triggered by hydrogen peroxide or superoxide anion under basic conditions; acridinium esters, which are triggered by hydrogen peroxide under alkaline conditions; 1,2-dioxetane derivatives (e.g., AMPPD, CDP-Star), which are triggered enzymatically by alkaline phosphatase (AP), which cleaves a phosphate group and initiates decomposition of the dioxetane ring; Ru(bpy)₃²⁺ (tris(2,2′-bipyridyl) ruthenium (II)), which is triggered electrochemically in the presence of a co-reactant such as tripropylamine (TPA); Firefly luciferin, which is triggered enzymatically by firefly luciferase in the presence of ATP, Mg2+, and molecular oxygen; Coelenterazine, which is triggered by Renilla luciferase or aequorin (calcium-dependent) in the presence of dissolved oxygen; and fluorescein (as fluorophore)+Diphenyl oxalate (DNPO) or Bis(2,4,6-trichlorophenyl) oxalate (TCPO), which is triggered by hydrogen peroxide in the presence of a base catalyst (e.g., sodium salicylate). The oxalate ester reacts with H₂O₂ to form a high-energy intermediate (1,2-dioxetanedione), which transfers its energy to the fluorescein dye, exciting it. The fluorescein then emits as it relaxes. Additional substitutes for fluorescein include rhodamine B, 9,10-diphenylanthracene, or rubrene which use the same triggering chemistry. It should be appreciated that some of the chemiluminescent species are actually chemiluminescent systems, some of which require multiple additional reactants, including the triggering substance. Accordingly, the use of the phrase “at least one chemiluminescent species” corresponds to at least one reactant of the chemiluminescent system. Further, the “at least one activated chemiluminescent species” corresponds to the energy-rich product of the chemiluminescent reaction. It is further appreciated by the person skilled in the art that the triggering substance is chosen based on the specific chemiluminescent system.

In some embodiments, the solution is in contact with two electrodes. In some embodiments, the solution is in contact with only one electrode. In some embodiments, the solution is not in contact with any electrodes. In some embodiments, the solution is an aqueous solution. In some embodiments, the solution comprises a water-miscible species such as methanol or ethanol. In some embodiments, the solution comprises electrolytes, some of which are dictated by the specific chemiluminescent system, for example, if the specific chemiluminescent system is intended to be an alkaline system, the electrolytes can comprise NaOH, KOH, NH₄OH, or the base known to work best with the specific chemiluminescent system. In another example, magnesium or calcium ions need to be used in the specific chemiluminescent system, for example, MgCl₂, Mg(NO₃)₂, CaCl₂, Ca(NO₃)₂, etc. Other electrolytes include NaCl, KCl, NH₄Cl, NaI, KI, and NH₄I.

In some embodiments, the metal nanoparticles comprise, consist of, or consist essentially of, at least one species selected from the group consisting of silver, gold, platinum, palladium, rhodium, ruthenium, osmium, iridium, copper, aluminum, nickel, chromium, zinc, and alloys thereof. In some embodiments, the metal nanoparticles comprise, consist of, or consist essentially of silver. In some embodiments, the metal nanoparticles comprise, consist of, or consist essentially of gold. In some embodiments, the metal nanoparticles comprise, consist of, or consist essentially of copper. In some embodiments, the metal nanoparticles comprise, consist of, or consist essentially of aluminum. In some embodiments, the film of isolated metal nanoparticles and the at least one chemiluminescent species are selected so there is spectral overlap between the emission of excited-state chemiluminescent species and plasmon absorption of the metal nanoparticles.

In some embodiments, the substrate comprises a species selected from the group consisting of glass, quartz, plastic, a semiconductor, paper, cellulose, cotton, nylon, silk, sapphire, diamond, noble metal films, wool fabrics, bank notes, indium tin oxide, metallic alloys or dielectric materials.

In some embodiments, the bias voltage is in a range of about 0.0001 V to about 1 V, or about 0.0001 V to about 0.001 V, or about 0.001 V to about 0.01 V, or about 0.01 V to about 0.1 V, or about 0.1 V to about 1 V. An advantage of the bias voltage is that the bias voltage dictates the anode and cathode, removes background noise, allows the magnitude of the current to be more easily determined, and allows for greater current signals to be realized/measured.

As disclosed herein, the median aspect ratio is in a range from about 1:1 to 1:6. The aspect ratio dictates the wavelength over which the metal nanoparticles will couple. Spheres (i.e., a 1:1 aspect ratio) only couple with a unique wavelength. In other words, the aspect ratio is chosen to couple different wavelengths of the electromagnetic spectrum. In some embodiments, the metal nanoparticles never have an aspect ratio of 3:2, whether identified as an average or a median.

In some embodiments, the electrodes comprise, consist of, or consist essentially of the same metal species as the metal nanoparticles. In some embodiments, the electrodes comprise, consist of, or consist essentially of the metal species that are different from the metal nanoparticles. In some embodiments, the electrodes can further comprise a polymer, a ceramic, glass, or combinations thereof, so long as the electrodes are electrically conductive.

In some embodiments, the current is detected and measured using an ammeter, oscilloscope, or a multimeter, as understood by the person skilled in the art. In some embodiments, the peak current is detected and measured, as understood by the skilled artisan.

In some embodiments, the induced current has less background noise, allows the magnitude of the current to be more easily determined, and has05 a greater current signal, than an induced current in the absence of the bias voltage.

In some embodiments, no external electromagnetic radiation source is used to activate any chemiluminescent species. In some embodiments, ultraviolet radiation (approximately 100-400 nm) is not used to activate any chemiluminescent species. In some embodiments, visible light (approximately 400-700 nm) is not used to activate any chemiluminescent species. In some embodiments, infrared radiation (approximately 700 nm-1 mm) is not used to activate any chemiluminescent species. In some embodiments, no far-field detector is to quantify emission. In some embodiments, none of the chemiluminescent species are intentionally tethered to the metal nanoparticles, either directly or indirectly, prior to the generation of CIPC. In some embodiments, none of the chemiluminescent species are intentionally tethered to another species, e.g., no antibodies or the like are labeled with chemiluminescent species. Advantageously, this means that the chemiluminescent species can flow or diffuse in and out of a preferred interaction region, which is about 2 nm to about 30 nm from the metal nanoparticles.

In some embodiments, a calibration system configured to calibrate the obtained induced current and determine one or more characteristics of the chemiluminescent species based on the calibrated current is used, as understood by the person skilled in the art.

The CIPC system and method described herein is used to detect a target molecule in a chemiluminescence assay, wherein the amount of target molecule can be quantified using a calibration curve and the detected induced electrical current. It should be appreciated that the CIPC system and method can also be used qualitatively to signal the presence of a target molecule without quantification of same. Chemiluminescence assays known in the art include, but are not limited to: chemiluminescent immunoassay (CLIA), which is a standard sandwich or competitive immunoassay wherein the detection antibody is labeled with a chemiluminescent species (e.g., acridinium esters or HRP/luminol); enhanced chemiluminescence (ECL) Western blot, which permits protein detection on a membrane using a HRP-conjugated secondary antibody with luminol/enhancer substrate, wherein the enhancer can be a substituted phenol like p-iodophenol; chemiluminescent ELISA, which is a conventional ELISA format substituting a chemiluminescent substrate (e.g., HRP/luminol or AP/dioxetane) for the standard chromogenic substrate (i.e., TMB); electrochemiluminescence immunoassay (ECLIA), which uses a Ru(bpy)₃²⁺-labeled antibody triggered at an electrode surface with TPA co-reactant; chemiluminescent nucleic acid hybridization assay, wherein the target nucleic acid is hybridized to a probe labeled with AP, detected using a dioxetane substrate; bioluminescent reporter gene assay, wherein cells are transfected with a luciferase reporter construct (firefly or Renilla) and luminescence is measured after cell lysis and substrate addition to quantify gene expression or promoter activity; dual-luciferase reporter assay, which combines firefly luciferase (luciferin/ATP) and Renilla luciferase (coelenterazine) in the same well, wherein the Renilla signal serves as an internal normalization control; chemiluminescence resonance energy transfer (CRET) assay, wherein energy from a chemiluminescent donor is transferred to a proximal fluorescent acceptor without an external light source; peroxyoxalate chemiluminescence (PO-CL) detection, typically coupled to HPLC as a post-column detection method. Analytes with native fluorescence (or fluorescently tagged) are detected via energy transfer from the oxalate/H₂O₂ reaction; and aequorin-based calcium assay, wherein aequorin (apoaequorin+coelenterazine) flashes upon binding intracellular Ca²⁺.

According, the method of generating and detecting a chemiluminescence-induced plasmonic current according to the first aspect can be used to detect a target molecule or biomolecule in a chemiluminescence assay, as understood by the person skilled in the art. Advantageously, the amount of target molecule or biomolecule can be quantified using a calibration curve and the detected induced electrical current. Target molecules or biomolecules to be detected and quantified are well known in the art of the skilled artisan working with assays and bioassays.

In other embodiments, the CIPC system and method can be used for any of the following: (1) imaging of chemiluminescent species within cells, bacteria, bugs and whole organisms, where the chemiluminescence originates either exo- or endogenously. For example, cells can be placed on a CIPC surface comprising the metal nanoparticles, and the location imaged and the induced current of any generated chemiluminescence detected and measured; (2) imaging the expression of proteins, DNA and RNA binding and/or hybridization; (3) as a new type of detector, where a far-field event causes near-field chemiluminescence which is then detected electrically through the CIPC mechanism. An example would be for the detection of alpha, beta or gamma radiation. These devices could either be stand-off devices or incorporated into wearable devices; (4) the imaging and detection of high energy solar/interstellar radiation, such as bursts of neutrinos or other high energy particles; (5) as a test for the analytical detection of either oxidizing or reducing agents. For example, an oxidizing agent comes into contact with a chemiluminescent species near-to the CIPC, and current is generated.

### EXAMPLE

To analyze CIPC, two reactions were used: (i) the luminol chemical reaction; and (ii) diphenyl oxalate, a compound that when combined with hydrogen peroxide produces 1,2-dioxetanedione, an unstable high energy intermediate that reacts with an inactive fluorophore to pump it to its excited state, which is the pathway for most commercially available glow sticks. In addition, a commercially available off-the-shelf luciferase ATP assay, which typically measures chemiluminescence emission to quantify ATP, but instead was tested using the CIPC system described herein, i.e., the assay was quantified by measuring the induced current, rather than the chemiluminescence emission.

### Materials and Methods

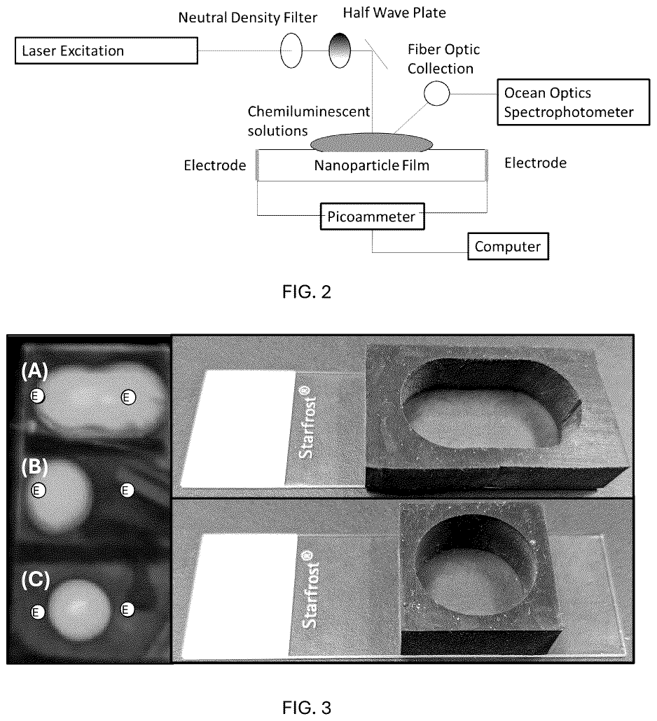

Important to the MEF, MEC and the FIPC systems is the use of plasmonically-active nanoparticles that share a strong absorption-emission spectral overlap with the desired luminescent species. For efficient energy transfer to occur from the excited-state species (donor) to the surface nanoparticles (acceptor), there should be strong spectral overlap between the emission of the excited-state species and the plasmon absorption of the nanoparticles. Due to the large wavelength range that is spanned by luminol (UV-blue range), luciferase (orange), fluorescein with diphenyl oxalate (Green), and sulforhodamine with diphenyl oxalate (Red), multiple different metal films were used. For the luminol and fluorescein reactions, 7 nm silver nanoparticle films were thermally deposited, and for the luciferase and sulforhodamine testing, 8 nm gold nanoparticle films were thermally deposited. Silver was chosen for luminol due to its larger spectral overlap in the blue-visual region, while gold was chosen to take advantage of the orange and red regions past 580 nm. Three nm copper films were also tested in some of the experiments to test against the cross reactivity of silver, i.e., as a control sample. Silver and copper films were produced through the use of thermal vapor deposition; the silver films using an Edwards 306 Auto evaporator, and the copper films using a Kurt J. Lesker thermal/E-Beam depositor at the Singh Center at the University of Pennsylvania. For both of these depositions, glass microscope slide substrates were mounted onto the evaporation collection plate in the device, and the vacuum was set relative to the machine, 2E-05 Torr for the Edwards model and 6E-06 Torr for the Lesker model. Once the desired vacuum was reached, the metal of choice was sublimated via an induced current heating method in a molybdenum boat, and the current applied was controlled such that the rate of deposition of the metal did not exceed 0.1 Å/s. It has previously been shown by the present inventor that controlling the rate to be below 0.1 Å/s leads to better nanoparticle isolation and the formation of an “island-like film” to help with the collection of the FIPC/CIPC signal [Moskowitz, J., et al., 2020]. The isolated nature of the nanoparticles better allows them to charge/discharge induced current/energy across the film. After production, the films were then electrically characterized as a quality control step using a voltage sweep method. While hooked up in series on a FIPC analysis experimental apparatus (FIG. 2), an increasing voltage is applied across the film. If the films are a continuous metal film, a linear relationship between increasing voltage and the resultant current is seen, as the resistance does not change (i.e., V=IR). The presence of a coulombic blockade in the current response or simply the presence of a staircase-like response to increasing voltage, indicative of a charge and discharge cycle across the film for different sized clusters of nanoparticles, electrically confirmed the presence of these isolated particles. These particles were additionally imaged via SEM to confirm their isolated nature and sizes and optically characterized via UV-Vis spectroscopy using a Cary 60 UV-Vis spectrophotometer. These spectra show film plasmon absorption, indicating the wavelengths that ideally overlap with the emission spectrum of the coupling fluorophore to induce FIPC or CIPC.

For the chemiluminescent solution preparations, the luminol (BIOSYNTH®) was prepared in an aqueous solution to a 1 mM concentration. The luminol solution was prepared with 1 mM of aqueous potassium ferrocyanide, to act as a source of iron for the activation of luminol. When the solution was ready for activation it was placed onto a silver nanoparticle film with silver electrical contacts in place (see, FIG. 2) and a catalytic amount of 3% hydrogen peroxide was added. The resulting change in induced current after activation was captured, as well as the emission spectra, using the experimental set-up of FIG. 2. For the diphenyl oxalate and sulforhodamine solutions, a 100 μM solution of sulforhodamine was initially produced, and to it was added a solution of 10 mM diphenyl oxalate, both prepared in a 50/50 (v/v) mixture of ethyl acetate and water. For the fluorescein solutions the same procedure was followed, but with a 100 μM concentration of fluorescein, instead of the sulforhodamine. The solutions were applied directly to the gold film (see, e.g., FIG. 2, but with gold film and gold electrodes) and activated with the addition of 3% hydrogen peroxide, at which point the resulting current change is collected. Finally for the luciferase-based ATP assay, a metabolism assay kit from ELABSCIENCE® was obtained, and the manufacturer's instructions followed to experimentally obtain the standard curve. This assay was initially designed for a microwell plate. The volumes were increased such that 200 μL of solution was present on the nanoparticle films in contact with both of the electrical leads. For this experiment the solution was activated prior to its application to the film, and as the luminescence intensity peaked around 5 min after activation, the solution-induced current response was monitored between 4.5 and 5.5 minutes after activation, and the highest observed current reported.

### Results and Discussion

### Surface Area Effects on the Chemiluminescence Induced Plasmonic Current Response

When one thinks about how chemiluminescence can be used to generate plasmonic current, it is important to realize that each individual nanoparticle plays a role in the overall generated current. Without being bound by theory, it is believed that if more nanoparticles are able to charge and discharge relative to the size of the film, there will be a higher induced current, i.e., that surface area would become a significant controlling factor when designing experiments. This is in contrast to FIPC where the excitation irradiance spot size is fixed. FIG. 3 details three different orientations that were studied, for both the fluorescein and diphenyl oxalate chemiluminescence pathways, having three different sample orientations labeled (A), (B) and (C), with (A) representing a 5 cm² area and (B) and (C) both representing 3 cm² areas, the only difference being that solution (B) is in contact with one of the electrodes while (C) is not. On the right side of FIG. 3, two neoprene sample isolators are shown, with the smaller of the two having a surface area of 3 cm² (bottom right) and the larger one 5 cm² (top right). The isolators were attached to the nanoparticle film of FIG. 2 with an adhesive prior to use.

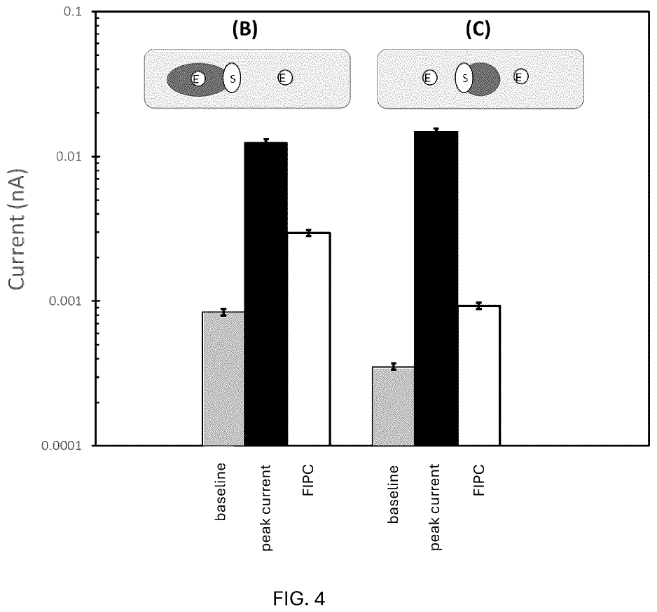

The first of the two orientations examined was the difference between (B) and (C), mainly because the difference between them is the presence of the electrode, and based on prior experimentation and literature [Moskowitz, J., et al., 2019; Pierce, D. R., et al., 2024], it has been shown that excitation areas closest to the electrode had a reduced resistance and a higher resulting current when excited for FIPC. This has previously been attributed to a reduced film distance over which the current flows. FIG. 4 details the experimental differences between (B) and (C). FIG. 4 is shown as 2 sets of 3 bars, each having a baseline current of the system after a standard bias voltage of 0.005 V was applied (“baseline”), a peak current obtained from the solutions of diphenyl oxalate and fluorescein after the introduction of hydrogen peroxide to catalyze the chemiluminescent reaction (“peak current”), and an FIPC response from the system which corresponds to the solution being excited using a 473 nm laser line after the chemiluminescent reaction had ended (“FIPC”). The two orientations are shown with matching labels from FIG. 3, as well as a small visual diagram of the solution relative to the electrodes “E,” and to the final excitation spot “S.” It can be seen in FIG. 4 that for the baseline current of the system, the inactive baseline is higher for the solution in contact with the electrode, i.e., (B), and this is also consistent from resistance readings showing lower resistance in that orientation, 5.95×10⁹Ω for (B) and 1.41×10¹⁰Ω for (C), per linear cm. Notably, the (C) FIPC response, i.e., the solution being optically excited after the chemiluminescent reaction has ended, is a similar trend relative to resistance values, with the solution for (B) giving a larger response at the same applied power, i.e., 10 mW, relative to (C), consistent with previous observations in FIPC experiments. Finally, and most interesting is that the peak current from both orientations appears to be very similar in magnitude to the other, with the orientation for (C) being only slightly higher than for (B). This finding raises a few new questions; 1) were the CIPC system responses not relative to the resistance of the system; and 2) was there actually a correlation between the surface area of the applied chemiluminescent solution and the induced current response of the system?

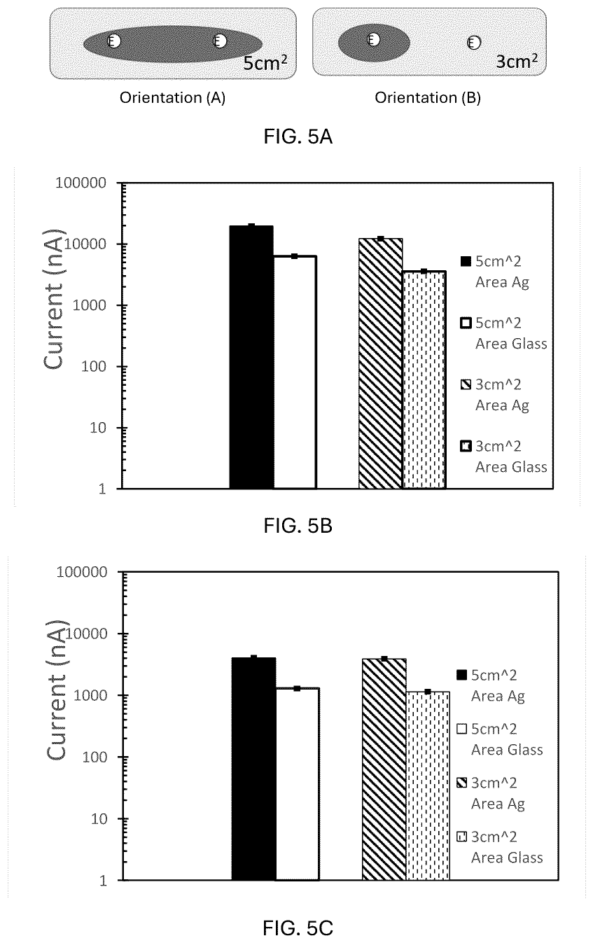

These questions were further explored in FIGS. 5A-5C, highlighting the differences between the orientation of solutions labeled (A) and (B). Since the previous experiment controlled for surface area and compared the results of resistance, the focus was shifted to the difference between the total area of the nanoparticle films covered by the diphenyl oxalate and fluorescein solutions. The solutions were placed on to the films within the isolators, consistent with the illustration of FIG. 5A, then activated by the addition of 3% hydrogen peroxide. To analyze the effects of the nanoparticle films in this process, and to serve as a control for CIPC, the experiments were also carried out on a series of bare glass slides that contained no plasmonic nanoparticles. FIG. 5B shows the response data for both the 5 cm² area and the 3 cm² area, and it can be seen that the 5 cm² area on both the silver film and the glass slide gave a higher response than the 3 cm² area for both. It was also noted that the responses on the silver film were found to be around three times higher than bare glass alone, ~20000 nA for the silver film and ~6300 nA for the bare glass peak current. An enhancement of 3.5 times was also noted for the 3 cm² surface area, ~1200 nA vs 3500 nA. This difference in current serves as a reminder that high energy chemiluminescent solutions show changes in resistance during their course of reaction, but not all of the current change noted is a result of this resistance; a significant portion is due to the CIPC. Both solutions show a decrease in resistance upon activation, which coupled with the applied bias voltage, leads to a current response in both the glass and the film systems. Further, it is interesting to compare the two surface areas relative to their unit area, i.e., the induced current response per cm², as shown in FIG. 5C. It can be seen that when the total surface area is taken into account (i.e., normalized for) the response between the two orientations is almost identical, ~4000 nA per cm² area. These findings are consistent with what had been noted for the volumetric effects of MEF, in that larger solution surface areas directly in contact with surface nanoparticles, produce a greater enhancement. This finding has further significance in that it implies that for both FIPC and its chemiluminescence analogue, CIPC, one would benefit from high surface area, “roughened” surfaces, where more fluorophores (excited-state species) have the ability to couple and interact with surface plasmon nanoparticles.

### Decay Rate Analysis of Chemiluminescence Induced Plasmonic Current

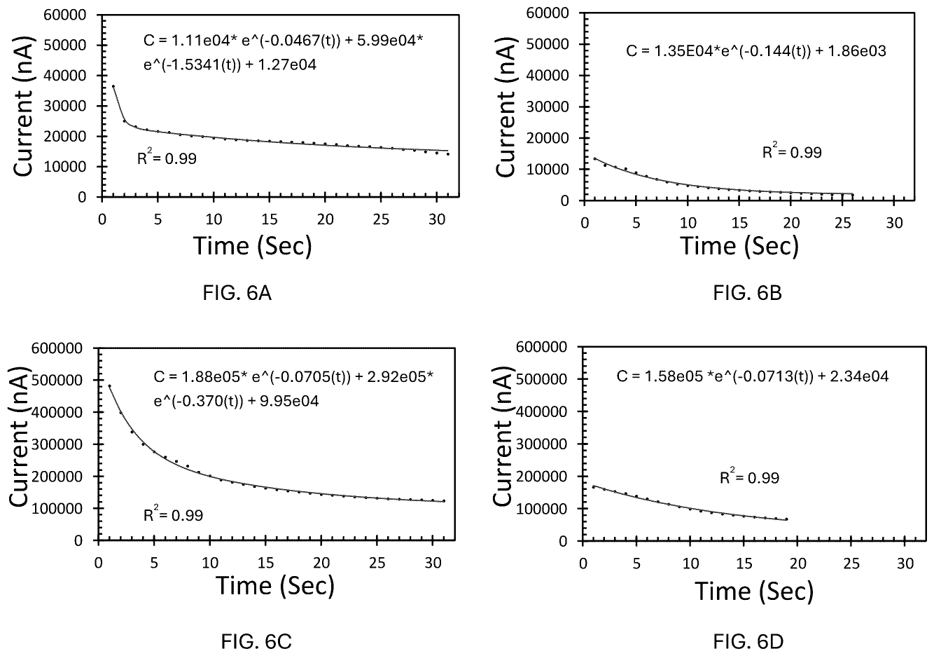

One of the main characteristics for both MEF and MEC is that when the excited-state species are in close proximity to the metal nanoparticles, not only is the overall intensity of the emission brighter, but there is a fundamental change in the lifetime/duration of intensity decay of the system. What is typically seen for MEC is that there is a bi-exponential intensity decay over time as compared to a mono-exponential decay seen in the absence of metal. To see if this process is similar for CIPC, two different sets of chemiluminescent solutions were studied, one containing sulphorhodamine and one luminol. The films and solutions were placed in series with a Keithley picoammeter, and activated, and the induced current was collected over time. To serve as controls, these experiments were also performed on bare glass slides. The results of this experiment can be seen in FIGS. 6A-6D, with further analysis of the data fitting found in Tables 1 and 2.

TABLE 1
Coefficients for the rates of induced current decay
for sulforhodamine/diphenyl oxalate solution on
the gold films of FIG. 6A and glass of FIG. 6B.

| | Current = Ae^{(-kt)} + A′e^{(-k′t)} + B | Current = Ae^{(-kt)} + B |
|---|---|---|
| | Gold Film | Glass |
| A | 1.11E−06 | 1.35E−06 |
| K (Amps/Sec) | 4.67E−02 | 1.44E−01 |
| B (Amps) | 1.27E−06 | 1.86E−07 |
| A′ | 5.99E−06 | |
| K′ (Amps/Sec) | 1.53E00 | |

TABLE 2
Coefficients for the rates of induced current decay for luminol
solution on silver films of FIG. 6C and glass of FIG. 6D.

| | Current = Ae^{(-kt)} + A′e^{(-k′t)} + B | Current = Ae^{(-kt)} + B |
|---|---|---|
| | Silver Film | Glass |
| A | 1.88E−05 | 1.58E−05 |
| K (Amps/Sec) | 7.04E−02 | 7.13E−02 |
| B (Amps) | 9.95E−06 | 2.34E−06 |
| A′ | 2.92E−05 | |
| K′ (Amps/Sec) | 3.70E−01 | |

FIGS. 6A and 6B both represent sulforhodamine and diphenyl oxalate solution, on the gold film and the glass substrate control sample, respectively. It can be seen that the overall response for the solution on the gold film is two times higher and that there is a rapid decay of current before leveling. Both single and bi-exponential decays were considered to model the decay of the induced current, for the single-exponential decay:

Current=Ae^{(-kt)}+B (Eq.1)

and for the bi-exponential decay:

Current=Ae^{(-kt)}+A′e^{(-k′t)}+B (Eq.2)

In Eqs. 1 and 2, tis time in seconds and B is a constant in Amps. At first glance, it can be seen that the induced current decay from the metal is not a mono-exponential function, whereas the decay on the glass control sample was much simpler to model, as evidenced by the fitting parameters shown in Table 1. From the fitting parameters, it appears that the current decay on gold may simply be the summation of the induced current (i.e., CIPC) plus the current induced from the chemiluminescence reaction itself, i.e., similar to the control glass substrate. That said, the decay constant K (in A/sec) on glass is approximately 3 times greater, i.e., 0.144 versus 0.0467 (Table 1), and this is attributed to parameterization during the fitting process.

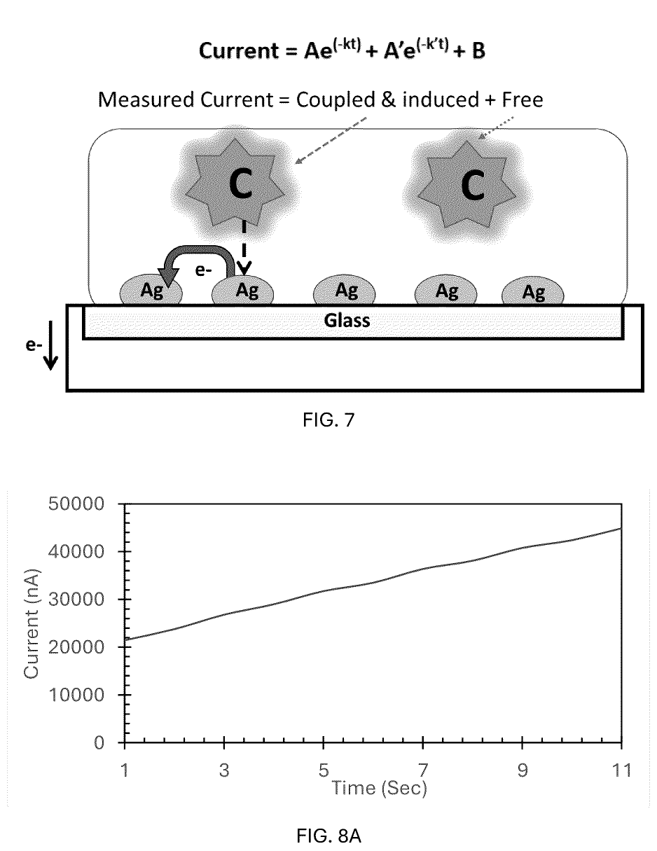

This analysis was also performed with the luminol solution on a silver nanoparticle film and a glass control slide, as shown in FIGS. 6C and 6D, respectively. It can be seen that there is a 2.5× current enhancement on the silver nanoparticle film, relative to the glass control slide, and that again a different rate of current decay rate from the nanoparticle film and the glass control substrate is seen, as shown in Table 2. Similar to what was noted from Table 1 and the parameters of the data fitting for FIGS. 6A and 6B, it was seen that the chemiluminescent solution on the metal nanoparticle films displayed evidence of the summation of these two current decays i.e., one rate on the metal and one rate from the glass. However, the rates of induced current decay K (in A/sec) were very similar, i.e., 0.0704 for Ag and 0.0713 for glass, respectively. FIG. 7 illustrates the decay of induced current from both metal and glass. Since the diphenyl oxalate/sulforhodamine and the luminol pathways have different excitation mechanisms, and yet both displayed similar current decay characteristics, it was reasoned that this was likely a direct result of the detection and subsequent decay of the chemiluminescence induced plasmonic current CIPC that was generated.

### Chemiluminescence Induced Plasmonic Current Characteristics

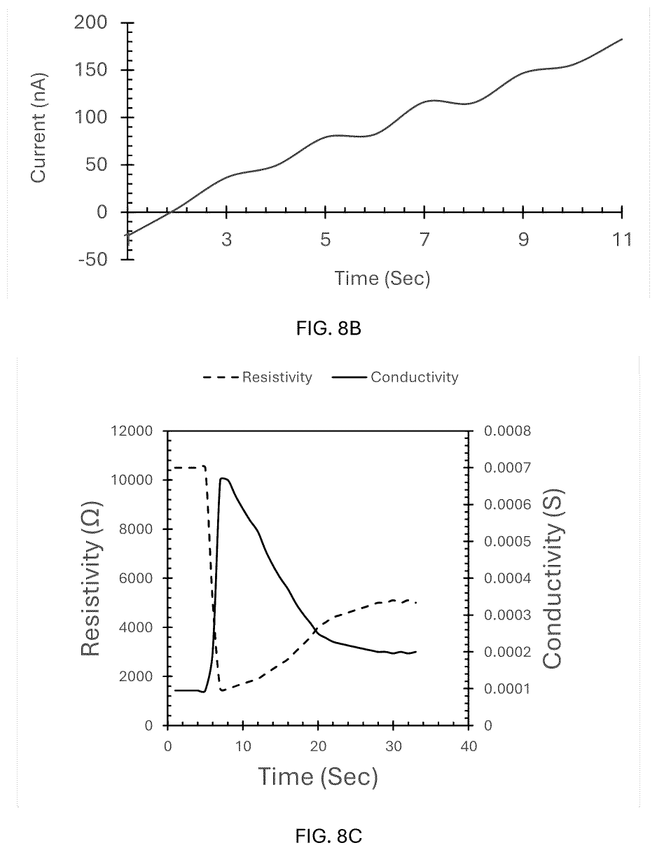

One of the techniques that has previously been shown useful in determining a nanoparticle films capacity for supporting FIPC is a voltage sweep experiment, wherein an increasing voltage across a film leads to a columbic blockade, or a staircase-like effect, in the current response. Accordingly, the electrical response of the luminol pathway, both during and post completion, was examined to see how the current responses would modulate, knowing that the luminol pathway and the degradation of 3-aminopthalate do not directly create any free electrons in solution. FIGS. 8A-8C detail this experiment, with the voltage sweep in FIG. 8A representing the active solutions, activated directly prior to the voltage sweep initiation, and FIG. 8B representing the solution after the light response and reaction had subsequently diminished. FIG. 8C shows the change in resistance of a comparable sample to illustrate changes in solution resistance. By comparing FIGS. 8A and 8B, it can be seen that there is a significant increase in the response of the system directly after activation and a significantly greater current than would be expected from the change in resistance/conductivity alone. From resistivity measurements, the change in current would have been equivalent to an 11× change in current, and after the initial drop in resistance, the resistance increases for the remainder of the reaction, which is not manifested in the electrical response of FIG. 8A. Accordingly, this experiment is more evidence suggesting that the chemiluminescent solution is providing a notable current to the film.

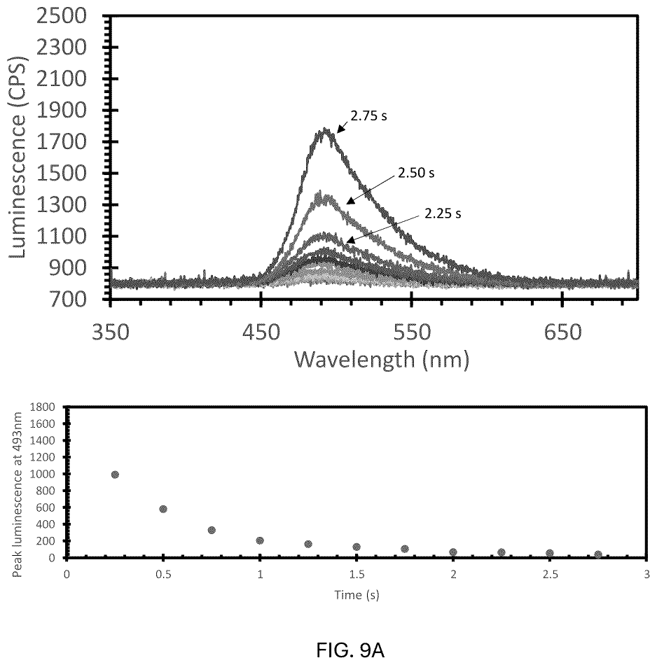

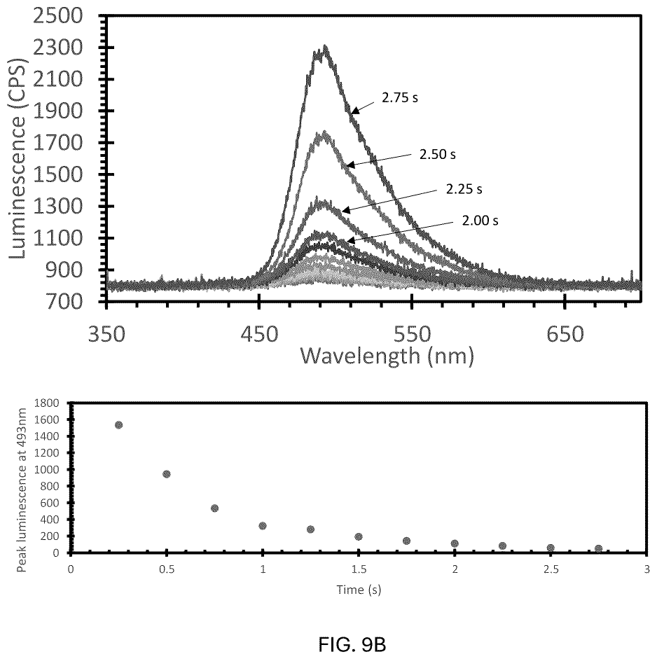

To further confirm this finding, voltage-gated experiments were performed on the solution-film combinations. Voltage-gated metal-enhanced fluorescence (VGMEF) experiments were originally used in the discovery and mechanistic interpretation of FIPC. In a typical MEF experiment the use of a high bias voltage (5 V) has the ability to “shut off” the metal enhancement of the emission, with the only emission seen during this being from the traditional far-field fluorescence emission alone. It is believed that the “shut off” is due to the surface nanoparticles having no ability to fluorophore couple when a high potential difference is forced across the nanoparticulate film. For the purposes of the present experiment, voltage-gated chemiluminescence was examined as a way to confirm that chemically induced electronic-excited states can couple and ultimately induce current in plasmonic films, i.e., CIPC. What has been noted in FIPC is that the overall far-field emission intensity of the coupled system actually diminishes as a portion of the energy is channeled to the film as induced electrical current rather than being emitted as light. FIGS. 9A-9B show the results of two different activations of luminol on 3 nm thick Cu films, one with a low bias voltage (0.005 V) typically used in FIPC experiments (FIG. 9A, top), and one with a high 5 V bias voltage to remove the coupling portion of the system (FIG. 9B, top). What can be seen between the two emission FIGS. 2.75 seconds after activation is that the film with the higher bias voltage has an overall increased luminescence relative to the much smaller bias voltage, and that both of these luminescence decays bear a very similar resemblance to the bi-exponential induced current decays shown in FIGS. 6A and 6C. As the bias voltage is increased dramatically, the nanoparticles on the film do not fluorophore couple and instead absorb, charge, and then discharge energy, ultimately resulting in a higher chemiluminescence far-field intensity. This finding strongly suggests that chemiluminescence signatures, similar to fluorescence, can induce surface plasmon currents.

### Chemiluminescence Induced Plasmonic Current ATP Assay

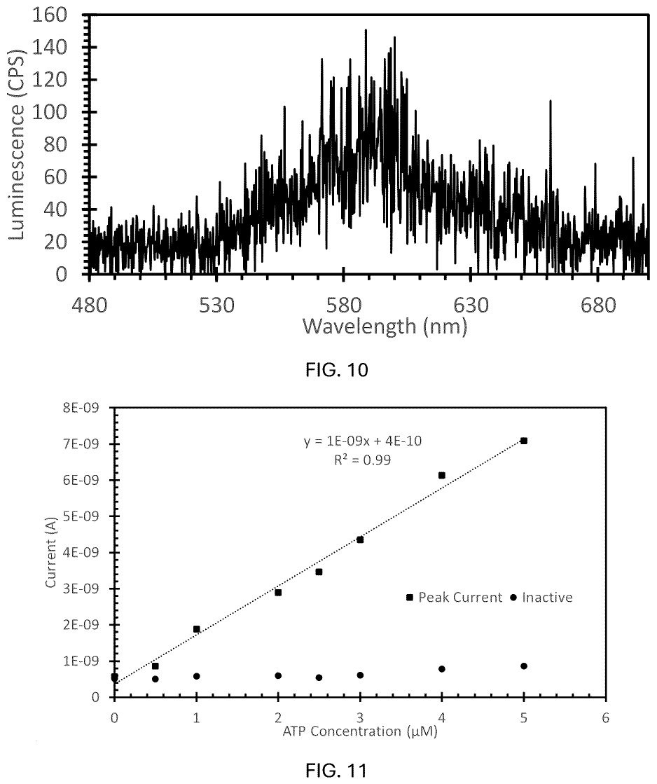

To demonstrate the utility of the CIPC approach, a commercially available chemiluminescent ATP-assay was applied to the CIPC platform, i.e., an ATP luciferase assay kit from ELABSCIENCE®. This style of luciferase assay is a markedly slower reacting one, the responses peaking around 5 minutes after initiation. To adapt this to the CIPC platform described herein, the desired solutions were activated in EPPENDORF® tubes, and applied to copper films 4.5 minutes after initial activation, where the peak current occurs between 4.9 and 5.1 minutes after activation. The standard curve provided was replicated, within the manufacturers recommended sensing range. To serve as a control, the solutions were permitted to fully inactivate and the peak induced current obtained from a similar time frame 15 minutes after activation, i.e., between 14.9 and 15.1 minutes after activation. This assay was initially designed to be performed in a microwell plate, however, it was adapted to the CIPC system by increasing the total reagents per test to ~200 μL, consistent with the other CIPC sample volumes tested. The solutions themselves were dim to the visible eye, so for the emission spectrum seen in FIG. 10, 200 μL was applied to a nanoparticle-lined QUANTAWELL™ plate to enhance the emission. FIG. 11 details the CIPC responses of the system and it can be seen that as the available amount of ATP present is increased from 0.5 μM to 5 μM, there is close to 7× increase in the reported CIPC current, and an effective linear detection range is achievable for calibration purposes. The difference becomes even more significant when you factor in the baseline current of the inactive solution, as this is the baseline current of the system based on the resistance of the solution itself, plus the added 0.005 V bias voltage that is used in this experiment to control electron flow. The responses achieved a good linearity with an R² value of 0.99. Also interesting is that these responses were achieved without a highly luminescent solution, further giving credence to using CIPC as a viable assay design platform.

### CONCLUSIONS

It was shown for the first time that chemically-induced excited states, i.e., chemiluminescence solutions, can similarly induce plasmonic current in metal nanoparticles deposited on substrates as far-field optically excited molecules, i.e., FIPC. CIPC was demonstrated using two well-known luminescent precursors, Luminol and diphenyl oxalate solutions, on three different nanoparticle substrates comprising silver, gold or copper. In addition to a demonstration of CIPC, other notable observations with regard to CIPC include, but are not limited to:
The volume and surface area of the chemiluminescence solution directly effects the magnitude of the induced current. It is likely that the surface area of the nanoparticle film plays much more of a significant contribution than does volume.
The decay of induced current (rate of current change with time) from the nanoparticle films comprises three components: 1) a dominant coupled current decay; 2) current resulting from uncoupled decay (i.e., the chemiluminescence solution from bare glass); and 3) a background current, which is applied to dictate the direction of current flow. This is depicted visually in FIG. 7.
The functions that describe the rate of induced current decay, i.e., Eqs 1 and 2, show that the amplitude of current decay from the gold surface is ~6× greater than the glass control, i.e., A vs A′ in Table 1, but only ~1.5× when we compared the decay on luminol induced-current from silver films, i.e., A vs A′ in Table 2.
The coulombic staircase is not clearly present with active chemiluminescence solutions, demonstrating that the solutions themselves are also contributing to current, in addition to the induced current via CIPC.
Voltage gating experiments for MNFs and chemiluminescence solutions demonstrate that the excited state molecules in chemiluminescence solutions are indeed coupling too and ultimately producing chemiluminescence induced plasmonic current.

Further, to demonstrate the practical utility of the CIPC approach, an ATP assay was demonstrated using a commercially available off-the-shelf kit. Remarkably, the chemiluminescence signatures which are usually detected by a photodetector to quantify the assay analytes of interest are simply determined here through surface current measurements, removing the need to use modestly expensive chemiluminescence plate readers. As disclosed herein, the chemiluminescence emission intensity from the actual assay solutions was very weak, but this has little bearing on CIPC measurements which are due to non-radiative energy transfer from the excited state species to surface metallic nanoparticles, i.e., radiative emission was not detected. Accordingly, it is envisaged that the multitude of chemiluminescence assays commercially available today can readily use the CIPC platform disclosed herein.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

Moskowitz, J.; Geddes, C. D. Plasmonic Electricity: Fluorophore-Induced Plasmonic Current. J. Phys. Chem. C 2019, 123, 27770-27777, DOI: 10.1021/acs.jpcc.9b08249
Moskowitz, J.; Sindi, R.; Geddes, C. D. Plasmonic Electricity II: The Effect of Particle Size, Solvent Permittivity, Applied Voltage, and Temperature on Fluorophore-Induced Plasmonic Current. J. Phys. Chem. C 2020, 124, 5780-5788, DOI: 10.1021/acs.jpcc.9b11276
Pierce, D. R., Saha, L., & Geddes, C. D. Fluorophore-induced plasmonic current generation from copper nanoparticle films. ACS Omega, 2024, 9 (23), 25181-25188, DOI: 10.1021/acsomega.4c02751.

What is claimed is:

1. A method of generating and detecting an induced chemiluminescence-induced plasmonic current (CIPC), said method comprising:
positioning a substrate comprising a film of isolated metal nanoparticles between two electrodes;
introducing a solution onto the film of metal nanoparticles, wherein the solution comprises at least one chemiluminescent species; and
activating the at least one chemiluminescent species with a triggering substance, wherein energy generated by the at least one activated chemiluminescent species nonradiatively transfers energy to the film of metal nanoparticles, resulting in an induced electrical current across the film; and
applying a bias voltage across the film and detecting the induced current.

2. The method of claim 1, wherein the induced electrical current of the at least one chemiluminescent species in the presence of the film of metal nanoparticles is enhanced relative to a control lacking the film of metal nanoparticles.

3. The method of claim 1, wherein the at least one chemiluminescent species and the triggering substance comprises a combination selected from: luminol and hydrogen peroxide or horseradish peroxidase; lucigenin and hydrogen peroxide or superoxide anion; acridinium esters and hydrogen peroxide; 1,2-dioxetane derivatives and alkaline phosphatase; tris(2,2′-bipyridyl) ruthenium (II) and tripropylamine; firefly luciferin and firefly luciferase; coelenterazine and Renilla luciferase or aequorin; or fluorescein plus diphenyl oxalate or bis(2,4,6-trichlorophenyl) oxalate and hydrogen peroxide.

4. The method of claim 1, wherein the metal nanoparticles and the solution are in contact with one or two electrodes.

5. The method of claim 1, wherein no external electromagnetic radiation source is used for activation.

6. The method of claim 1, wherein no far-field detector is to quantify emission.

7. The method of claim 1, wherein the metal nanoparticles comprise at least one species selected from the group consisting of silver, gold, platinum, palladium, rhodium, ruthenium, osmium, iridium, copper, aluminum, nickel, chromium, and zinc.

8. The method of claim 1, wherein the metal nanoparticles have a median diameter in a range of from about 3 nm to about 500 nm.

9. The method of claim 1, wherein the metal nanoparticles have a median aspect ratio in a range of from about 1:1 to about 1:6.

10. The method of claim 1, wherein the median distance of metal nanoparticle to metal nanoparticle is about 2 nm to about 250 nm apart.

11. The method of claim 1, wherein the substrate comprises a species selected from the group consisting of glass, quartz, plastic, a semiconductor, paper, cellulose, cotton, nylon, silk, sapphire, diamond, noble metal films, wool fabrics, bank notes, indium tin oxide, metallic alloys or dielectric materials.

12. The method of claim 1, wherein the film of isolated metal nanoparticles and the at least one chemiluminescent species are selected so there is spectral overlap between the emission of excited-state chemiluminescent species and plasmon absorption of the metal nanoparticles.

13. The method of claim, 1, wherein the bias voltage is in a range of about 0.0001 V to about 1 V.

14. The method of claim 1, wherein the electrodes comprise the same the metal species as the metal nanoparticles.

15. The method of claim 1, wherein the electrodes comprise different metal species from the metal nanoparticles.

16. The method of claim 1, wherein the induced current has less background noise, allows the magnitude of the current to be more easily determined, and has a greater current signal, than an induced current in the absence of the bias voltage.

17. The method of claim 1, wherein the method of generating and detecting a chemiluminescence-induced plasmonic current is used to detect a target molecule or biomolecule in a chemiluminescence assay.

18. The method of claim 17, wherein the amount of target molecule can be quantified using a calibration curve and the detected induced electrical current.
